(19) United States
(12) Patent Application Publication
(10) Pub. No.: US 2004/0236202 A1
(43) Pub. Date: Nov. 25, 2004

(54) Expandable strap for use in electrical impedance tomography

(76) Inventor: Steven Angell Burton, Midlothian, VA (US)

Correspondence Address: John H. Thomas, P.C., 1561 East Main Street, Richmond, VA, 23219, US

(21) Appl. No.: 10/444,012

(22) Filed: May 22, 2003

(51) Int. Cl. A61B 5/04

(52) U.S. Cl. 600/384; 600/390; 600/393

(57) ABSTRACT

An assembly that incorporates an expandable strap for use in electrical impedance tomography includes a strap adapted to be mounted onto the body of a patient. A plurality of electrodes is mounted on the strap and spaced equidistantly along the length of the strap. An electrical connector is mounted on the strap, and electrical traces extend from each electrode to the connector. Preferably, the strap is made of a substantially inelastic material. In one embodiment, the strap has a serpentine shape to allow for uniform expansion.

The field of the invention is an electrode assembly for use in electrical impedance tomography. More specifically, the invention is an assembly that holds a plurality of electrodes and maintains uniform spacing of those electrodes during their placement and use on a subject.

### BACKGROUND OF THE INVENTION

Electrical impedance tomography (hereinafter “EIT”) involves the production of images representing the distribution of an electrical characteristic, such as electrical conductivity or resistivity, across a sectional plane of a body under investigation from measurements made on the periphery of the sectional plane. The technique may be used for the noninvasive investigation of human patients as well as the investigation of other objects or bodies. It is a relatively inexpensive method of tomography, allows continuous monitoring, and does not suffer from biological hazards implicit in other procedures such as X-ray computed tomography.

EIT involves the application of spaced electrodes to the surface of a body under investigation, usually in the form of a line around the body, such that the electrodes lie in the plane of the body to be investigated. In a typical biomedical EIT system, low voltage alternating electrical current is applied between two neighboring electrodes with the resulting potentials measured between pairs of all the remaining electrodes. The measured values from all such potential measurements are stored and processed to create a two-dimensional image of the resistivity distribution within the body. A static image may be created, showing the absolute value of tissue resistivity, or a dynamic image may be produced, displaying the changes in resistivity from a reference. The dynamic image is clinically useful as changing features of the body such as cardiac activity and lung activity may be monitored.

In order to obtain useful results, it is important that the electrodes are spaced equidistantly around the object being monitored. When a plurality of electrodes is used, for instance sixteen electrodes in many preferred applications, it is difficult to easily space those electrodes apart accurately and keep them substantially in a plane.

Furthermore, an electrode assembly should not disrupt or impede normal respiration. This is of particular importance given that many potential applications of EIT involve the use on patients with compromised or dysfunctional respiratory drive.

Finally, an electrode assembly that can be easily taken on and off is advantageous in that the patient may be required to wear the assembly for many hours and even days. Quick applications of the electrode assembly will provide a substantial benefit to the patient care process.

### SUMMARY OF THE INVENTION

Accordingly, it is an object of the present invention to overcome the foregoing drawbacks and to provide an assembly that incorporates a strap, and preferably an expandable strap for use in EIT that can be easily and reliably placed around a patient. When using a substantially inelastic elastic material as a strap, the expansion may be obtained through use of a serpentine shaped strap or through use of a strap that is releasably overlapped onto itself at regular intervals around the strap.

In one embodiment, an expandable strap for use in electrical impedance tomography includes a strap adapted to be mounted onto the body of a patient. The strap is comprised of a substantially inelastic material. A plurality of electrodes are mounted on the strap and spaced equidistantly along the length of the strap. An electrical connector is mounted on the strap, and electrical traces extend from each electrode to the connector. The strap has a serpentine shape. The serpentine shape may be a uniform wave. The inelastic strap material may comprise a polymer sheet. That polymer sheet may comprise polyester. The electrical traces may comprise conductive ink.

In a still further embodiment, an expandable strap for use in electrical impedance tomography includes a strap adapted to be mounted onto the body of a patient. The strap is comprised of a substantially inelastic material. A plurality of electrodes are mounted on the strap and placed equidistantly along the length of the strap. An electrical connector is mounted on the strap, and electrical traces extend from each electrode to the connector. Each of the portions of the strap between each adjacent pair of electrodes is overlapped onto itself and releasably adhered to itself, and further each overlapped strap portion will release at substantially the same rate from a tension that may be applied along the length of the strap. The expandable strap may further comprise a plurality of sleeves, when a sleeve is wrapped around each of the portions of strap that is overlapped onto itself, and further wherein the sleeve does not prevent the release of the overlapped portions. The substantially inelastic material may comprise a polymer sheet. That polymer sheet may comprise polyester. The electrical traces may comprise conductive ink. A strap may be coated with an adhesive material to releasably adhere the strap to itself in the overlapped strap portion.

In another embodiment, an electrode assembly for use in electrical impedance tomography includes a strap adapted to be mounted onto the body of a patient. The assembly further comprises a plurality of electrodes mounted on the strap and spaced equidistantly along the length of the strap. The assembly also includes an electrical connector mounted on the strap and electrical traces that extend from each electrode to the connector. The strap may be expandable. The strap may be adapted to wrap around the body of a patient. The strap may be comprised of a plurality of strap segments.

In a still further embodiment, the invention includes a method of mounting an electrode assembly onto a patient for use in electrical impedance tomography. The method includes the steps of providing an electrode assembly comprising a strap adapted to be mounted onto the body of a patient, a plurality of electrodes mounted on the strap and spaced equidistantly along the length of the strap, an electrical connector mounted on the strap, and electrical traces that extend from each electrode to the connector. The method further includes the step of measuring the diameter of a patient's body around the portion of the body where the assembly will be mounted. The method includes stretching the strap so that the length of the strap substantially equals the measured diameter of the patient. Finally, the method includes wrapping the strap around the patient when the electrodes are contacted with the body of the patient. The method may further include the step of measuring the diameter of the patient's body using a measuring tape. Still further, the electrode assembly may comprise an integral measuring tape.

### BRIEF DESCRIPTION OF THE DRAWINGS

FIGS. 1A and 1B are schematic diagrams demonstrating alternative embodiments of the present strap assembly invention.

FIG. 2 is a top plan view of an expandable strap in accordance with the present invention.

FIG. 3 is a side elevation view of the strap shown in FIG. 2.

FIG. 4 is a side elevation view of the strap shown in FIG. 2 with the strap in an extended position.

FIG. 5A is a perspective view of a strap in its ordinary unextended position.

FIG. 5B is a perspective view of the strap shown in FIG. 5A except that the strap is in an extended position.

FIG. 6 is a perspective view of an alternative embodiment of the present invention.

FIG. 7 is a side elevation view of the strap shown in FIG. 6.

FIG. 8 is a side elevation view of a still further embodiment of the strap shown in FIG. 6.

FIG. 9 is a side elevation view of the strap shown in FIG. 8 in an extended position.

FIG. 10 is a schematic diagram of an electrode assembly with unique identifier circuitry.

### DETAILED DESCRIPTION OF THE PREFERRED EMBODIMENTS

The present invention is an economical and efficient electrode assembly to be used in conjunction with electrical impedance tomography (EIT). The electrode assembly includes a strap (of one or more strap segments) having electrodes mounted on it. The strap may be formed from inexpensive material yet, at the same time, its construction is preferably adapted to expand in a uniform manner such that the electrodes mounted on the strap will be mountable equidistantly from one another. This uniform spacing of the electrodes is important for effective results in EIT. The components are economical enough that the assembly can be disposed of after use and a new assembly used for a subsequent patient.

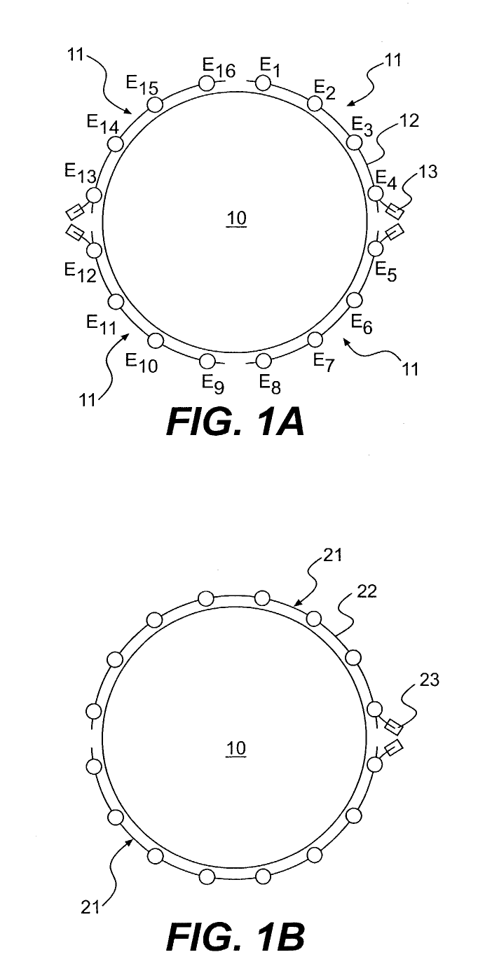

FIGS. 1A and 1B are schematic diagrams demonstrating two alternative embodiments of the present invention and how it may be applied. FIG. 1A shows a body 10 having four separate strap segments 11 mounted around its perimeter. Each strap segment 11 has four electrodes (E1-E16) attached to it. The strap segment 11 is comprised of an actual strap 12 having a connector 13 mounted on one end of the strap. As shown in FIG. 1A, the strap segments 11 are mounted around the body 10 so that the connectors 13 are oriented to the right and left sides of the body 10. In this way, other leads (not shown) that are attached to the EIT equipment are merely led from the patient on the patient's two sides. The body 10 is shown ideally as a circle. Obviously, the body 10 could be the body of a patient, the head of a patient, or any other object that is able to be subjected to EIT. As is demonstrated in FIG. 1A, each of the electrodes is spaced equidistantly around the circumference of the body 10.

FIG. 1B demonstrates an alternative embodiment of electrode assemblies or strap segments 21 having a strap 22 and connector 23. Each strap segment 21 contains eight electrodes such that only two straps are needed to be fixed around the circumference of a body 10. The connectors 23 are aligned adjacent each other so that the leads to the EIT equipment only extend on one side of the patient or object being examined.

Further alternatives include a plurality of straps, each having multiple (two or more) electrodes, that are electrically connected to each other in series. This “daisy chain” construction would provide for the strap segments to be electrically connected (or connectable) to each other so that only one connector (or at least less than one connector for each strap segment) is necessary as the actual lead to the EIT equipment. Of course, different and/or supplemental circuitry would be required for the straps and connectors to allow for this “daisy chain” alternative. For instance, connectors with more prongs and straps that have more electrical leads (conductive traces and/or wires) would be necessary to account for the different signals detected and/or sent from each electrode.

As shown in FIGS. 1A and 1B, a preferred set-up for EIT includes 16 electrodes. While this is a common layout, an EIT analysis may include additional electrodes or fewer electrodes. The number of electrodes is driven by the size of the body being measured and the desired precision of the measurements. It is also driven by the software that is written to actually operate the EIT process. It may still be further affected by the sensitivity and versatility of the electrodes that are used in the construction. The illustrated assemblies (and similar assemblies with different numbers of electrodes) of the present invention may be-used in connection with any of these alternative constructions that require that the electrodes be spaced apart substantially equidistantly around the body being examined.

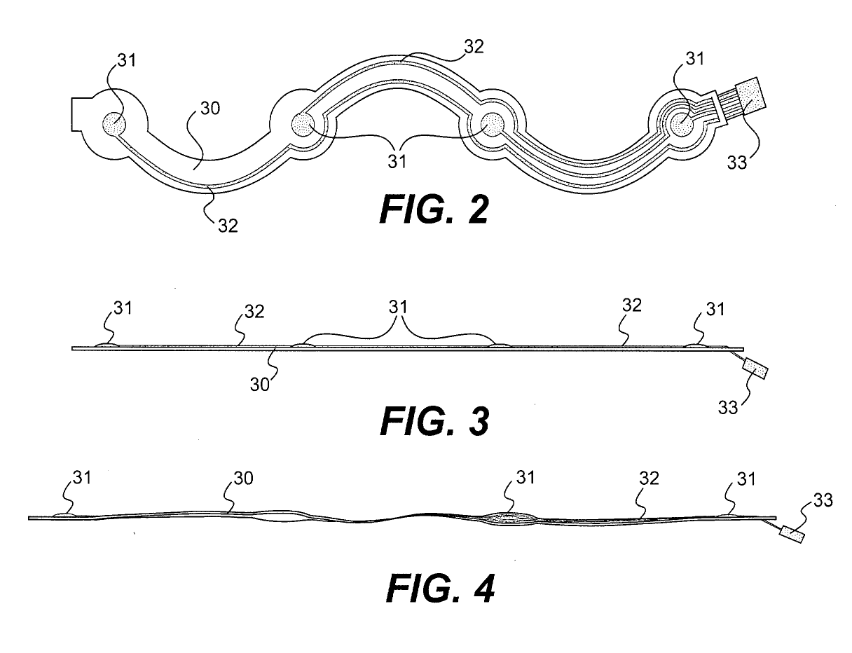

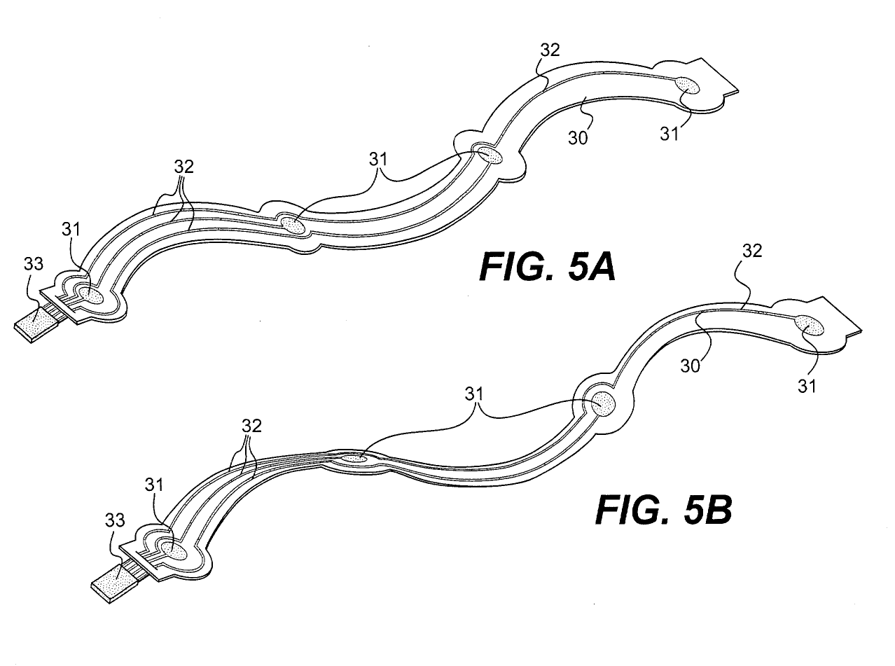

FIGS. 2-5B demonstrate one preferred embodiment of an electrode assembly with a uniformly expandable strap for use in EIT. FIGS. 2, 3 and 5A illustrate in various views the strap 30 in its ordinary, unextended position. FIGS. 4 and 5B illustrate the strap 30 in an extended or expanded position.

Referring now to FIGS. 2-5B, strap 30 is a flat substrate onto which are mounted electrodes 31. The electrodes 31 are spaced substantially equidistantly along the length of strap 30. Electrical traces 32 extend from each electrode 31 to the connector 33. The connector 33 is a conventional, four prong connector adapted to receive or be inserted into a reciprocal connector that goes to the EIT apparatus that sends and picks up signals through a body by way of the electrodes 31.

In a preferred embodiment, the strap 30 is made of a Mylar (polyester) film having a nominal thickness of 0.005 in (0.127 mm). It is important for the invention that the strap 30 be made of a material that is flexible and bendable, yet inelastic. The strap 30 may be made of many types of polymer films such as polyesters, polypropylenes, etc. The strap 30 may also be made of, for instance, a woven material. The strap 30 shown in the figures is substantially flat. For the purposes of this preferred embodiment of this invention, it is important that the strap substrate has the integrity and bias to maintain a serpentine shape at rest on a plane and that can support the electrodes mounted therein. The strap itself may have different thicknesses and rounded structure for all or part of its construction. A plastic film as noted is a preferred structure because of its low cost.

It is also beneficial, when the electrodes are mounted on a patient, that the assembly supporting the electrodes be made of an expandable material or have an expandable structure to allow for the electrodes to maintain their proportional spacial relationship around the circumference of the body of the patient during the inhalation and exhalation phases of respiration. Constriction of respiration is uncomfortable and could result in inaccurate readings.

The electrodes 31 may be made from any conventional construction of electrode. Typically, the electrode 31 would be a silver/silver chloride-based construction. An electrode similar to the electrode described in co-pending application Ser. No. 10/121,541 entitled SENSOR FOR BIOPOTENTIAL MEASUREMENTS, filed Apr. 12, 2002, may be used. That application is incorporated herein by reference as if set forth in its entirety. As noted, however, virtually any electrode may be mounted on the strap 30 as may be necessary or desirable in the EIT procedure. The electrodes 31 are connected to the connector 33 by way of electrical traces 32. The traces 32 extend from each electrode 31 to the connector 33. In a preferred embodiment, conductive ink is used as the electrical trace. The conductive ink is literally printed onto the surface of the strap 30. For this reason, the strap 30 cannot be so stretchable that the conductive traces 32 would be interrupted and signals lost. Of course it is possible for the traces 32 to be made from other materials that may or may not be attached directly to the strap 30. For instance, the traces 32 could embody separate thin wires that connect each electrode 31 to the connector 33. The electrical traces 32 of conductive ink are merely an economical and reliable alternative.

As shown in FIGS. 4 and 5B the strap 30 may be extendable. Physically, the strap 30 will buckle as shown to allow it to be extended. As illustrated, the strap 30 is in the serpentine shape of a uniform wave (FIG. 2). In this way, as the strap 30 is extended, the electrodes 31 remain spaced apart equidistantly, all be it farther apart from each other than in the original, unextended condition. The uniform wave shape demonstrated is not the only serpentine shape that could be used. Of course, other types of waves or bends in a strap such as strap 30 could be used to allow the electrodes to inherently space apart equidistantly as the strap is extended—either during application onto or respiration by a patient.

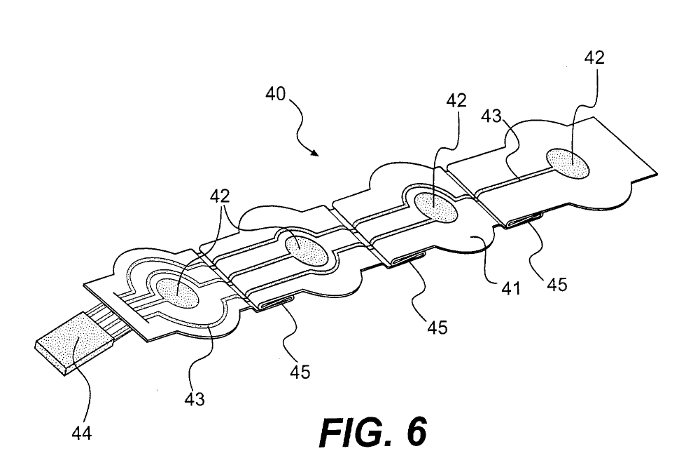

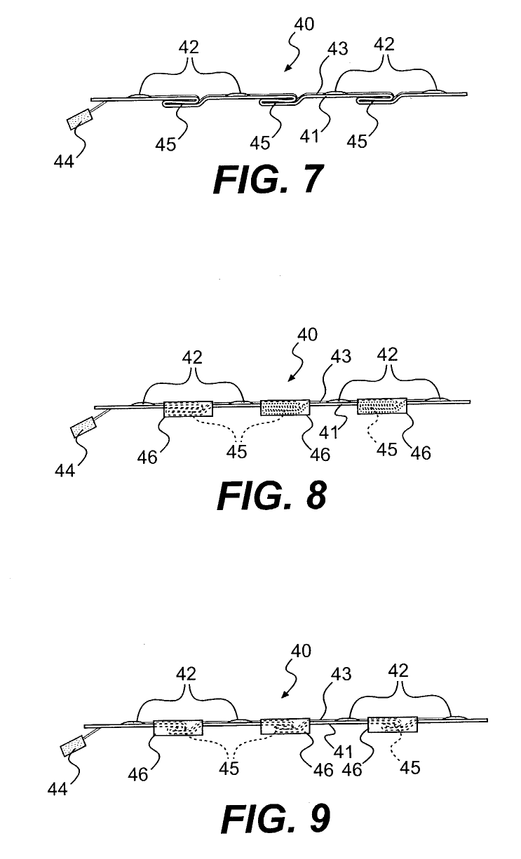

FIGS. 6-9 demonstrate an alternative preferred embodiment of the present invention. The electrode assembly 40 is made up of a substantially flat strap 41 onto which are mounted electrodes 42. The electrodes 42 are connected to a connector 40 by way of electrical traces 43. The portions of the strap 41 between the electrodes 42 are shown overlapped into portions 45. These overlapped portions 45 are releasably adhered to themselves in order to maintain the assembly 40 in a stable condition. However, when the electrode assembly 40 is pulled on either end, the assembly will extend or expand through the release of the overlapped portion 45. Importantly, the overlapped strap portions 45 will release at substantially the same rate from a tension that may be applied along the length of the strap 41. In a preferred embodiment, the overlapped portion 45 is releasably adhered to itself through use of an adhesive such as a silicone pressure sensitive adhesive.

FIGS. 8 and 9 demonstrate a further alternative to this second embodiment. The electrode assemblies 40 shown in FIGS. 8 and 9 further include sleeves 46 that are wrapped around the overlapped portions 45. The sleeves 46 merely encapsulate the overlapped portions 45. The sleeves 46 still allow for the release of the overlapped portions. Additionally, the sleeves 46 may be engineered to hold the overlapped portions 45 together so that they will release in a uniform rate as the assembly 40 is extended. The sleeves 46 may be an elastic material. Alternatively, it may be a rigid construction. It is only necessary that the sleeves 46 allow for a uniform rate of extension between electrodes 42 when the assembly 40 is being extended. An adhesive may or may not be necessary when sleeves 46 are used. In other words, the sleeves 46 may themselves be used to control the release of the overlapped portions 45.

The foregoing illustrations FIGS. 2-9 are directed to strap assemblies having four electrodes. As noted earlier, the assemblies may include more or less than four electrodes. The teachings noted herein would apply equally to longer or shorter straps having a different number of electrodes.

Clinically, one way to facilitate the placing of a strap on a patient is to wrap a tape measure around a patient or other object being measured in order to determine the exact diameter around which the electrodes will be mounted. Then, in the example of straps comprising four electrodes and an EIT system requiring sixteen electrodes, the tape that measures the diameter of the body is then folded in half twice in order to get an accurate measurement of the length of one quarter of the body being measured. That length may then be used to extend the specific, four-electrode straps and mount them accordingly on the patient. Alternatively, the assemblies described herein may include an integral measuring tape that facilitates the specific lengthening of the strap or strap segments to be appropriate for a given patient or other body being analyzed. This alternative could be a two-piece assembly with one serpentine strap as discussed and a second, non-extendable cord (the guide measuring tape) loosely woven into the strap or connected by few loops to the strap. The guide tape could be used as noted to set the proper length of the electrode strap. The guide tape could even then be discarded.

The electrical circuitry associated with preferred embodiments of an electrode assembly may also be enhanced. Referring for example to FIG. 6, although any electrode assembly could incorporate these attributes, one or more of the connector 44, electrical traces 43, or electrodes 42 could be modified to make each electrode have a unique electrical signal. Most simply, each electrode 42 would have a different electric resistence value than the other electrodes. This allows the equipment that drives the EIT process to differentiate between the multiple electrodes 42. Other embedded electrical signals or differentiators could be used to help a controller running the EIT process to send proper signals to the proper electrodes and better interpret signals received from the electrodes. As noted, any one or more of the connector 44, electrical traces 43 or even the electrodes 42 can be modified to create the unique signals.

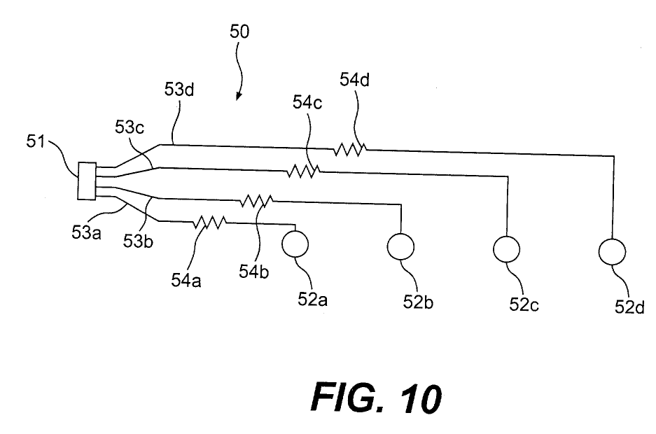

FIG. 10 is a schematic diagram of an electrode assembly 50 with the unique identifier circuitry imbedded in the electrical traces. The assembly 50 has a conventional connector 51 electrically connected to electrodes 52a-d through corresponding electrical traces 53a-d. Each of the traces 53a-d incorporates a corresponding, distinctive resistor 54a-d. The different and distinctive resistors 54a-d allow the EIT equipment to specifically identify each electrode 52a-d.

Another possible modification to the electrical circuitry would be an electronic “key” to insure proper electrical connections and prevent, for instance, the improper attachment of the wrong types of electrodes. This modification may be made to a connector 44, electrical trace 43 or electrode 42. The “key” feature could be distinctive resistance imbedded in one or more of the foregoing components. A connector 44 could also be mechanically manipulated to be distinctive such that only compatible electrode strap segments could be joined together or connected to the EIT equipment.

While the invention has been described with reference to specific embodiments thereof, it will be understood that numerous variations, modifications and additional embodiments are possible, and accordingly, all such variations, modifications, and embodiments are to be regarded as being within the spirit and scope of the invention.

What is claimed is:

1. An expandable strap for use in electrical impedance tomography, the strap comprising:
a strap adapted to be mounted onto the body of a patient, the strap comprised of a substantially inelastic material;
a plurality of electrodes mounted on the strap and spaced equidistantly along the length of the strap;
an electrical connector mounted on the strap;
electrical traces that extend from each electrode to the connector;
wherein the strap has a serpentine shape.

2. The expandable strap as described in claim 1, wherein the serpentine shape is a uniform wave.

3. The expandable strap as described in claim 1, wherein the substantially inelastic strap material comprises a polymer sheet.

4. The expandable strap as described in claim 3, wherein the polymer comprises polyester.

5. The expandable strap as described in claim 1, wherein the electrical traces comprise conductive ink.

6. An expandable strap for use in electrical impedance tomography, the strap comprising:
a strap adapted to be mounted onto the body of a patient, the strap comprised of a substantially inelastic material;
a plurality of electrodes mounted on the strap and spaced equidistantly along the length of the strap;
an electrical connector mounted on the strap;
electrical traces that extend from each electrode to the connector;
wherein each of the portions of the strap between each adjacent pair of electrodes is overlapped onto itself and releasably adhered to itself, and further wherein each overlapped strap portion will release at substantially the same rate from a tension that may be applied along the length of the strap.

7. The expandable strap as described in claim 6, further comprising a plurality of sleeves, wherein a sleeve is wrapped around each of the portions of the strap that is overlapped onto itself, and further wherein the sleeve does not prevent the release of the overlapped portions.

8. The expandable strap as described in claim 6, wherein the substantially inelastic strap material comprises a polymer sheet.

9. The expandable strap as described in claim 8; wherein the polymer comprises polyester.

10. The expandable strap as described in claim 6, wherein the electrical traces comprise conductive ink.

11. The expandable strap as described in claim 6, wherein the strap is coated with an adhesive material to releasably adhere the strap to itself in the overlapped strap portion.

12. An electrode assembly for use in electrical impedance tomography, the assembly comprising:
a strap adapted to be mounted onto the body of a patient;
a plurality of electrodes mounted on the strap and spaced equidistantly along the length of the strap;
an electrical connector mounted on the strap; and
electrical traces that extend from each electrode to the connector.

13. An electrode assembly as described in claim 12, wherein the strap is expandable.

14. An electrode assembly as described in claim 12, wherein the strap is adapted to wrap around the body of a patient.

15. An electrode assembly as described in claim 14, wherein the strap is comprised of a plurality of strap segments.

16. The expandable strap as described in claim 1, wherein one or more of the plurality of electrodes, electrical connector or electrical traces further comprises distinctive electrical circuitry for identifying each electrode.

17. An expandable strap as described in claim 6, wherein one or more of the plurality of electrodes, electrical connector or electrical traces further comprises distinctive electrical circuitry for identifying each electrode.

18. An electrode assembly as described in claim 12, wherein one or more of the plurality of electrodes, electrical connector or electrical traces further comprises distinctive electrical circuitry for identifying each electrode.

19. A method of mounting an electrode assembly onto a patient for use in electrical impedance tomography comprising the steps of:
providing an electrode assembly comprising a strap adapted to be mounted onto the body of a patient, a plurality of electrodes mounted on the strap and spaced equidistantly along the length of the strap, an electrical connector mounted on the strap, and electrical traces that extend from each electrode to the connector;
measuring the diameter of a patient's body around the portion of the body where the assembly will be mounted;
stretching the strap so that the length of the strap substantially equals the measured diameter of the patient; and
wrapping the strap around the patient wherein the electrodes are contacted with the body of the patient.

20. A method as described in claim 19, wherein the step of measuring the diameter of a patient's body includes using a measuring tape.

21. A method as described in claim 20, wherein the electrode assembly further comprises an integral measuring tape.
